(19) United States
(12) Patent Application Publication
(10) Pub. No.: US 2005/0049453 A1
(43) Pub. Date: Mar. 3, 2005

(54) Hydraulically driven vibrating massagers

(76) Inventor: Roger W. Faulkner, Brighton, MA (US)

Correspondence Address: Brian M. Dingman, Esq.;Mirick, O'Connell, DeMallie & Lougee, LLP, 1700 West Park Drive, Westborough, MA, 01581-3941, US

(21) Appl. No.: 10/925,552

(22) Filed: Aug. 25, 2004

Related U.S. Application Data
(60) Provisional application No. 60/497,385, filed on Aug. 25, 2003.

(51) Int. Cl. A61F 5/00

(52) U.S. Cl. 600/38

(57) ABSTRACT

The invention is a means to vibrate and/or rhythmically deform various devices for massage and/or sexual stimulation, and the devices themselves, comprising at least two, and normally three elements: (1) one or more hydraulically driven oscillators within a massage and/or sexual stimulation device; (2) a hydraulic actuator to move hydraulic fluid into and out of the device (1) (3) optionally, a flexible hose or hoses connected at one end to the sexual stimulation device or devices and at the other end to the hydraulic actuator. The particular devices actuated by the present invention include various massage and/or sexual stimulation devices, including muscle massagers, dildos, and penis rings (with or without attachment to secondary anal stimulators), sexually stimulating underwear, and two-ended dildos intended to anchor on one end (the wearer's end) in the wearer's vagina and optionally also in the wearer's anus, for example. For purposes of this disclosure, “sexually stimulating devices” and other similar language should also be understood to include massage devices that are not specifically intended for sexual stimulation. Compared to prior art vibrating sexual stimulators, the devices of this invention are capable of more complex modes of motion and both higher amplitude and slower deformation rates. The hydraulically actuated sexual stimulators of this invention are powerful, compact, quiet, and have low rates of heat generation compared to prior art electrically-driven vibrating sexual stimulation devices. In addition, said devices can be conveniently heated or cooled via the hydraulic fluid.

### CROSS REFERENCE TO RELATED APPLICATION

This application claims priority of Provisional application Ser. No. 60/497,385, filed on Aug. 25, 2003.

### FIELD OF THE INVENTION

Several kinds of erotic stimulators are known in the prior art, such as:
vaginal dildos for females;
penile masturbators;
anal dildos for males and females
penis rings for males and couples;
underwear designed to be worn during sex.
underwear designed to excite the wearer while being worn during non-sexual activities.
   Examples of each of these devices that include an electrically powered vibrator are known in the prior art. Devices that combine two or more of these functions are also known in the prior art.

### BACKGROUND OF THE INVENTION

The human penis becomes and remains erect by hydraulic pressure. Therefore it is useful to consider some hydraulic characteristics of the penis as a sort of prior art dildo. A review of the workings of the penis is contained in the August, 2000 issue of Scientific American, in an article by Irwin Goldstein on “Male Sexual Circuitry.” During erection the muscles in the penis artery walls relax, allowing blood to flood in to the porous sinus-like tissue. The penis inflates to a pressure nearly equal to the systolic (arterial) blood pressure of the male, as the porous sinus-like tissue of the penis inflates with blood during an erection. The pressure inside the penis changes slightly with the beating of the heart, but this is usually not perceptible to the male's sexual partner. The actual pressure inside the porous sinus-like tissue of the penis is on the order of only 200 torr (the torr is the conventional unit of blood pressure, also known as “millimeters of mercury”), or around one fourth to one third of atmospheric pressure.

During ejaculation, there is a special deformation of the penis, which many people find extremely exciting sexually. This deformation is caused by muscle contractions, the flow of the ejaculate, and changing penile inflation pressure during ejaculation. No prior art dildo is known which can accurately simulate the feel of an ejaculation to the sexual partner of the one ejaculating, though several attempts have been made.

Three recent patents on novel dildos include U.S. Pat. Nos. 4,790,296, 5,725,473, and 5853362. In U.S. Pat No. 4,790,296, an electric motor supplies rotary energy to a gearbox that translates the rotational motion of the drive shaft into reciprocating motion of an elongated arm along its longitudinal axis. This allows for the motor and gearbox to be remote from the dildo, a desirable goal since the motor and gearbox are relatively heavy, and the motor needs to be ventilated for cooling. The devices of U.S. Pat. No. 4,790,296 involve a long, rigid sexual stimulator arm that necessarily couples to a gearbox. The dildo of U.S. Pat. No. 4,790,296 can reciprocate at any realistic rate, including very slow movements, by putting a gear reducer between the drive motor and the gearbox. However, the device in U.S. Pat. No. 4,790,296 is not convenient, in that the rigid stimulator arm limits the range of motions that are possible for the user or users. The device in U.S. Pat. No. 4,790,296 seriously limits sexual motions, and tends to decrease sexual innovation.

U.S. Pat. No. 5,725,473 describes “a sexual aid including a housing, mounted on detachable legs and containing a motor that urges a dildo, including vibration means, to describe an arcuate path generally coincident with an orifice, such as a vagina.” This device is cumbersome, in that it is not possible for one sexual participant to use the stimulator while simultaneously and flexibly interacting with a sexual partner. The devices of U.S. Pat. No. 5,725,473 are only minimally useful in sex between partners when at least one of those partners is a male.

U.S. Pat. No. 5,690,603 on an erogenic stimulator describes a two-sided dildo that is anchored in either a vagina or an anus by the contraction force of the anal sphincter or the vaginal wall muscles. Tightness in these muscles hold the dildo as the user engages in sex with a partner, thus dispensing with any need for straps to hold the dildo in place. This patent is also related to several U.S. design Pat. No.: 384,158, 384,156, 376,650, and 320087. This patent mentions the possibility of having a hollowed-out area in the base of the dildo that can hold a vibrator for increased stimulation. Alternately, a fluid chamber within the apparatus can allow the flow of fluid to increase stimulation. This patent and its related design patents do not disclose or anticipate variation of the pressure of the flowing liquid.

U.S. Pat. No. 5,853,362 describing a “glandular stimulator device and method” provides a unique method to anchor a two-sided dildo into a woman's vagina, by using a hook-shaped flexible dildo as an anchor. This anchoring can be supplemented by an additional connection to an anal stimulator. This device is then normally used to stimulate a partner's vagina, anus, or prostate with a second external dildo. This patent mentions the desirability of adding vibration to the anchoring hook-shaped dildo or the attached external dildo, though the concept of vibration is discussed only in a general sense. This patent does not disclose or anticipate the use of rapid variations of inflation pressure as a means of causing vibrations.

U.S. Pat. No. 4,476,880 describes an apparatus for sensing and indicating vaginal muscle contractions. In this device, the pressure of a gas or a hydraulic fluid inside a dildo-like device with flexible rubbery walls changes as the vaginal muscles contract, thus providing quantitative data on the strength of the contraction that can be used in physical therapy, for biofeedback training, or for research. This patent does not disclose or anticipate the use of the flexible-walled dildo in conjunction with a hydraulic actuator to form a sexual stimulator.

U.S. Pat. No. 4,574,791 describes a muscle-toning device that is used to exercise vaginal muscles. This patent describes a hollow dildo-like device that contains a slidable mechanism that is moved back and forth by vaginal contractions of the user. This patent does not disclose or anticipate the actuation of said slidable mechanism by a hydraulic actuator to form an externally driven sexual stimulator.

U.S. Pat. No. 4,167,938 describes an exerciser for vaginal muscles that incorporates an air-inflated bladder which is linked to a rigid core. The purpose of the rigid core in this dildo design is to restrict the expansion of the bladder to being purely radial. This patent does not disclose or anticipate the use of liquid for inflating said bladder, nor does it anticipate rapid changes of inflation pressure being used to generate exciting motions or vibrations of the dildo.

### DETAILED DESCRIPTION

Hydraulically driven rhythmically deforming sexual stimulators are disclosed which may operate by means of varying inflation pressure in one or several fluid-filled tubes and/or sacs inside the hydraulically driven sexual stimulators. Compared to prior art vibrating mechanisms in which the vibration is driven by a rotating asymmetric weight, the hydraulically driven rhythmically deforming sexual stimulators of the present invention are capable of imposing stronger rhythmic deformations with a wider range of cycle time than prior art electrically driven vibrators. The amplitude or “strength” of the deformation is independent of the frequency, which is not the case for prior art vibrators. Alternatively, the hydraulically actuated sexual stimulators of this invention can also contain vibrators based on rotating imbalanced mass rotors driven by hydraulic motors.

Varying the pressure within various sexually stimulating inflated tubes and sacs can provide sexual stimulation in several different ways. Devices can be designed that are useful for sex with a partner (all sexual preference pairs can be accommodated), or for masturbation, for either a man or a woman. Several embodiments of this invention can be mounted in lingerie or underwear. The devices mounted in lingerie or underwear could either be designed to cause orgasm for the person wearing it, or to excite and stimulate without causing orgasm. In the non-orgasmic mode, devices of the present invention can enhance sexual pleasure for pairs of lovers, increasing the intensity of mutual orgasm.

Specific examples of the devices of this invention can accomplish each of the following multifunctional sexual stimulation modes:
1) simultaneous vaginal and clitoral stimulation
2) anal stimulation while leaving the genitals free for sex;
3) clitoral stimulation while leaving the vagina free for sex;
4) simultaneous anal and clitoral stimulation while leaving the vagina free for sex;
5) simultaneous vaginal, clitoral, and anal stimulation while orally stimulating a partner's genitals;
6) vaginal stimulation while leaving the clitoris free for oral sex;
7) anal stimulation while leaving the clitoris free for oral sex;
8) simultaneous anal and vaginal stimulation while leaving the clitoris free for oral sex;
9) anal stimulation while leaving the penis free for oral sex;
10) prostate stimulation while leaving the penis free for oral sex;
11) simultaneous anal, prostate, and/or scrotal stimulation while leaving the penis free for oral sex;
12) penile stimulation while leaving the anus free for sex;
13) vibrating the erect male penis during sex;
14) vibrating, squeezing, or massaging the breasts during sex;
15) vibrating, squeezing, or massaging the testicles and/or scrotum during sex;
16) stimulating either a man or woman while that same person is engaged in oral sex with a partner, in such a way as to leave both hands free.

Compared to prior art methods to generate a slow, reciprocating motion of a dildo to simulate coitus, the varying hydraulic pressure dildos of the present invention are intrinsically safer, and their safety can be easily improved by the application of well understood methods for limiting hydraulic pressure. In addition to their improved safety, this method can also create the desired motion while allowing the user greatly improved flexibility in both position and orientation. See for comparison prior art patents such as U.S. Pat. Nos. 4,790,296 and 5,725,473, which rely on eccentric gears and/or pistons. Note that eccentric gears and pistons present the danger of potential pinch points. Hydraulically driven sexual stimulators avoid these potential pinch points, and in addition offer a dramatically reduced risk of injury due to electric shock.

A dildo of the present invention can use time-varying average hydraulic pressure in internal tubes within the dildo to change the hardness level of said dildo, which mimics nature in regard to the slowly increasing erection state of the dildo; this comes close to simulating the erection of the penis. The exerciser of U.S. Pat. No. 4,167,938, if modified to be inflated by a liquid hydraulic fluid, is perhaps the simplest example of the present invention, albeit a non-preferred design.

An advantage of using a hydraulic drive to vibrate a sexual stimulator rather than an electric motor coupled to an imbalanced rotating weight (the most common prior art method) is that the hydraulic device can be much quieter than an eccentric rotating mass vibrator.

Another advantage of using a hydraulic drive to vibrate a sexual stimulator rather than an electric motor is that the hydraulic device can run much cooler than an electrically driven vibrator. In fact, the fluid pumped into and out of the sexual stimulator can be used for heating or cooling the stimulator if desired. This aspect of the invention can be applied to conventional eccentric rotating mass vibrators that are driven by hydraulic motors.

Another advantage of using a hydraulic drive to vibrate a sexual stimulator rather than an electric motor is that the hydraulically driven device (whether driven by inflation/deflation or by a hydraulic motor) can weigh less and be smaller than a vibrating electric motor with similar power. Only a relatively thin hydraulic hose or pair of hoses needs to connect the sexual stimulator to the hydraulic power pack. Therefore it is feasible to wear the device while remaining mobile and movable over the bed or other area used for sex.

### PREFERRED EMBODIMENTS

There are three ways to achieve vibrations or other rhythmic motions within a sexual stimulator via movement of hydraulic fluid into and out of said sexual stimulator per the present invention. One option is to vary the pressurization of various flexible sacs and or tubes, or a hydraulic cylinder within the sexual stimulator. In this method fluid flow is normally back-and-forth, rather than continuous (though the basic design can be modified to cause a net flow of fluid through the device, as is desirable for temperature control). Second, a fluid flowing through the device can power a hydraulic motor that drives a conventional vibrator, such as an imbalanced rotor vibrator, an eccentrically driven piston, or a twisted shaft, for example. Third, a flowing fluid can provide direct stimulation through vibrations generated by fluid turbulence, as in the prior art U.S. Pat. No. 5,690,603.

One group of sexually stimulating devices per this invention are various dildos, which will be discussed momentarily. Novel sexual stimulating devices that are pleasurable for males as well as their partners can also be devised per the present invention, which vibrate the erect penis. This type of device might be used by couples who would be unlikely to use most other types of sexual stimulators. This can be accomplished for example by moving hydraulic fluid into and out of one or more fluid-inflated sacs or flexible cavities that apply a force to the base of the erect penis as the male engages in sex with a partner. Such sacs would typically be held in place by a specially designed harness, which could look like modified leather underwear.

The simplest possible design for a hydraulically activated dildo of this invention is a hollow elastomeric dildo filled with hydraulic fluid. As the hydraulic driver (which is discussed separately later) alternatively inflates, then deflates such a dildo, a rhythmic deformation at the same frequency as the fluid pressure cycle is felt by the user. In the simplest case of a pure elastomer tube with uniform wall thickness, the longitudinal strain is nearly equal to the radial strain, which is probably not the optimum for sexual stimulation. The shape change of a hollow dildo of the present invention due to the inflation/deflation cycle can be varied greatly by design features of the dildo, especially the placement of reinforcing fibers, wires, annular rings of high modulus material, or other known means to produce anisotropic mechanical properties in the dildo wall. It is anticipated that highly individualized dildos could be made analogous to the way that eye glasses are tailor-made at low cost, even though the process is fairly complex.

By placing circular loops of high-modulus material (such as fiber loops or wire rings) within the elastomeric wall, the dildo's deformation can be made almost purely longitudinal. Such a dildo can be hand-held, or it and can be discretely linked to an undergarment, such as leather underwear. This type of radially constrained dildo deforms primarily by elongating. When held fixed at one end to a wearable harness or specially designed underwear, such as leather or elastomeric underwear, the wearer would feel as though she/he was actually being fucked, or very nearly so. The frequency of the deformation and even the details of the thrust rate during each moment of the cycle, could be adjusted/controlled by means known in the prior art. (For example, feedback controls like those used to control the motion of the piston in injection molding machines, and simpler mechanical/hydraulic devices like eccentric gears or cams to control pistons that are connected to the sexual stimulation device.) The frequency for the cyclic hydraulic fluid motions of the dildo can be quite slow (e.g., less than 0.1 hertz) compared to a much higher minimum practical frequency for prior art vibrating dildos based on rotating eccentric masses (the most common prior art method).

Such a hydraulic dildo can also contain a device known within the prior art to impart relatively high frequency vibrations, as discussed below. Said secondary vibration can be generated via a pressure oscillation that is imposed by an oscillating cylinder or bellows that is physically located inside the hydraulic control unit, at some remove from the sexually stimulating device per se. The hydraulic driver in this case would apply both a low frequency deformation to change the shape of the dildo and also apply a second, higher-frequency, lower amplitude oscillation that can be in the audible range (30-20,000 hertz) via a fluid-immersed electrical coil-driven diaphragm, piezoelectric crystal, or by other known means. In this scenario, the high & low frequency drivers operate independently, and can be independently adjusted, though both can be positioned within the hydraulic driver assembly. The frequency response range for the main hydraulic fluid motions into and out of the dildo are relatively low (e.g., about 0.1-20 hertz) compared to a much higher frequency response of 5-20,000 Hz for the second oscillator. Both the high and low frequency responses of such a dildo (including both intensity and frequency) are readily adjustable unlike the commonly available vibrating dildos of the prior art. The average hydraulic pressure can also be varied to simulate increasing excitement, as occurs with a penis.

The sexual stimulating devices of this invention can incorporate one or more electrically-driven vibrating devices, such as a conventional electrically-driven eccentric mass vibrator, an electromagnetically driven diaphragm, or an electronically-driven piezoelectric crystal, that are inside the sexual stimulating device.

The novel method of deforming portions of a sexual stimulator can be applied to prior art sexual stimulators, such as the combination anal dildo/erection ring of U.S. Pat. No. 5,387,179. Such penis-vibrating devices can also be hybridized with a hydraulic means to stimulate the prostate gland, for example with a hooked dildo to stimulate the prostate from the inside the anus, as described in U.S. Pat. No. 5,853,362, or by rhythmically-variable pressure applied from outside the anal canal (via an inflating/deflating flexible cavity or sac that is in contact with the perineum between the scrotum and the anus), or even by gripping the prostate gently between two inflatable tubes so designed that they close linearly or circularly around the prostate, in such a way that the grip varies in a stimulating manner. This could again be combined with previously described means of applying higher frequency vibrations to the same area.

Hydraulically driven vibrating dildos of the present invention can be engineered to undergo a great variety of deformations when hydraulic fluid moves into and out of one or more fluid-inflated sacs inside the dildo. This principle can also be extended to the double-ended dildos of U.S. Pat. No. 5,853,362.

Hydraulically driven vibrating dildos of the present invention can be engineered to achieve a wave-like deformation that is highly suggestive of a male orgasm, as fluid cascades through a specially-designed series of inflatable elastomeric chambers. This method entails a series of elastomeric chambers which are separated by pressure activated valves, and so designed that fluid initially pumped into Chamber 1 is transferred to Chamber 2, and so on through the entire series of inflatable chambers. For this to work, the wall stiffness of Chamber 1 must be higher than the wall stiffness of Chamber, and so on through the sequence of chambers. Chamber 1 is rapidly inflated with a specific volume of fluid (by the hydraulic driver, though a relatively high pressure hose), which causes Chamber 1 to inflate, and the pressure to rise high enough to open the first pressure relief valve (Valve 1), between Chamber 1 and Chamber 2. Once Valve 1 opens, the first chamber partially deflates as it inflates the next chamber, Chamber 2, because the wall stiffness of Chamber 2 is lower than that of Chamber 1. As Chamber 2 is inflated by the fluid injected from Chamber 1, the pressure rises until Valve 2 opens (at a lower pressure than Valve 1). This process is repeated through a sequence inflatable chambers, each with lower wall stiffness than the previous chamber, and each equipped with a pressure relief valve that opens at a lower pressure than the previous valve in the sequence. The last pressure relief valve in the sequence allows a volume of fluid approximately equal to the injected fluid volume to return to the hydraulic actuator by a separate, relatively low pressure hydraulic hose. (This motion can also be simulated by a pulsating flow of a liquid, inside an elastomeric “skin” on the outside of the dildo.)

Hydraulically driven dildos per the present invention can be engineered to deform in ways that no living penis can deform and at frequencies that can be readily adjusted by the user. Said deformations include both low and high frequency vibrations, large amplitude motions of tens of centimeters and various twisting and turning motions that would be very difficult and/or cost prohibitive to activate by electromagnetic means.

The exerciser of U.S. Pat. No. 4,167,938, if first inflated by a non-oscillatory hydraulic drive to a desired average stiffness chosen by the person using it as a dildo, could then be partially inflated/deflated rapidly by hydraulic fluid via an oscillatory hydraulic driver; this would be a desirable example of the present invention.

Hydraulically driven vibrating dildos of the present invention can be engineered to consist of multiple layers, for example a molded rubber core coated by latex rubber or a polyurethane coating. In this case, it would be desirable to have variable wall and/or layer thicknesses to generate time varying bumps, ridges or sideways motions during hydraulic pressure cycling.

There are several interesting kinds of hydraulic dildo deformations that are essentially impossible to produce with isotropic layered dildo walls, even if multiple layers of uneven thickness are used. For example, using strictly isotropic materials for the dildo wall, it is difficult to engineer a hydraulic dildo such that elongates without increasing its diameter during fluid injection. However, by using high modulus fibers imbedded in the rubber, it is possible to create a dildo that deforms in this way. Thus, particularly preferred materials of construction for hydraulically driven dildos are elastomers with imbedded fibers and/or fabric having much higher modulus than the elastomers.

Various surface coatings and textures can be used on the sexual stimulators of this invention. Silicone rubber, various known polyurethanes that simulate the properties of human skin, and numerous other elastomers can be used in principle. Important properties such as stiffness, strength, fatigue resistance, and frictional properties can be adjusted to a large extent via formulation of the elastomer, for each potential type of elastomer. Other properties, including sterilizability, resistance to oil swelling, and biocompatibility are difficult to address though formulation of additives alone, and require picking certain specific grades of elastomer.

### HYDRAULIC DRIVER OPTIONS

Any means by which hydraulic fluid can be alternately injected and then removed from the sexual stimulator to generate a cyclic deformation therein may be used to drive the hydraulic sexual stimulators of this invention. A particular example of a hydraulic driver is a cylinder, which is moved by an eccentric, attached to a rotating shaft, thus injecting and removing hydraulic fluid in a pattern where deformation and flow are sine waves.

More complicated hydraulic drivers are also possible, by using cams or computer-controlled drivers (such as those presently used in controlling plastic injection molding machines); using such hydraulic drivers, cyclic deformations that are not simple sine waves can be created. Computer-controlled hydraulic drivers produce a time-varying displacement of a hydraulic cylinder or bellows, which is directly proportional to a voltage output from a computer-based controller. One particularly simple device for converting electrical energy to a controlled hydraulic deformation is a voice-coil driver. This method consists of a solenoid type coil directly coupled to the shaft of a piston. The coil and piston mechanism is in turn coupled to a spring, which provides a base level of pressure. Low frequency alternating current is applied to the coil, which in turn drives the shaft, driving the piston. Hydraulic fluid is driven into and out of the piston, moving the stimulator. This method has the benefit of having a faster response time than other approaches. Due to its high slew-rate, it is capable of applying arbitrary waveforms up to a maximum (cut-off) frequency dependent on the construction of a particular device.

In one particularly simple embodiment of the invention, a dildo which includes fluid filled tubes is so designed that the hardness of the dildo is adjustable by the user; such a dildo need not include any additional means to vibrate the dildo. In this case, a simple syringe pump could suffice for pressurizing the dildo.

One particular embodiment of the invention uses a voice coil, a D/A (Digital-to-Analogue) converter, and a PIC (Programmable Interface Controller), to generate a time-varying hydraulic pressure central to the present invention. This hardware can optionally be contained in a handheld unit powered by batteries. This mechanism is flexible enough that audible tones can be used for the hydraulic stimulation (for example).

The exact means by which hydraulic fluid is moved into and out of the sexual stimulator is not an essential feature of this invention.

Illustrative Examples of Sexual Stimulators of the Invention

### EXAMPLE 1

### Moderate Cost Hydraulic Driver Based on Rotary Electric Motor to Power the Drive

This example focuses on the hydraulic drive portion of the devices of this invention. The following briefly describes the key elements of an economical hydraulic drive that can be applied to any of the sexual stimulator/massage devices of this invention:
(1) Bellows rather than hydraulic cylinder minimizes maintenance, leakage potential, and initial cost of hydraulic driver;
(2) Variable speed motor allows user to control frequency;
(3) Adjustable eccentric arm allows user to vary stroke amplitude;
(4) Pressurization of hydraulic fluid reservoir by air from manual pump allows convenient control of average pressure in sexual stimulator.
   Example 1 notes
   The bellows should be oriented vertically to prevent bubbles from being trapped therein.
   The reservoir should be above the bellows to “prime the pump” with hydraulic fluid.
   Water is the preferred hydraulic fluid.

### EXAMPLE 2

### Simple Elastomeric Hydraulic Appliance

In this example an elastomeric vibrating appliance, which could be a muscle massager, dildo, cock ring, clitoral stimulator or etc., consisting of one or more hollow, fluid-filled, interconnected sacs composed of a single crosslinked elastomeric composition is prepared by conventional rubber molding methods. Said appliance is molded to be smaller than the fully inflated dimensions, as is true with any inflatable article. In the simplest implementation (relevant primarily for dildos), this appliance is simply inflated with liquid to give a desired hardness; this can be done with a simple pump or even by attaching it to a pressurized water supply; inflating with warm or cold water could supply desirable sensations for some users. When activated by cyclic varying hydraulic pressure, as for example by the mechanism of Example 1, said appliance expands and contracts with a frequency and amplitude determined by the hydraulic driver. The deformation of this appliance is both radial and longitudinal. The relative radial expansion is approximately equal to the relative lengthening of the appliance during the fluid cycle.

### EXAMPLE 3

### Elastomeric Hydraulic Appliance With Electrical Vibrator

The appliance of this example is essentially identical to that of Example 2, but is supplied with an electrical vibrator within the appliance. The speaker is used to impart high frequency vibrations to the appliance that cannot be conveniently obtained by hydraulics alone. The vibrator is electrically driven, and can be any of several designs, including wide frequency response speakers, imbalanced rotors tuned to specific frequencies, piezoelectric crystals, and other means. This vibrator can be activated by a variety of electronic signals, ranging from a single frequency form to complex music or biofeedback generated signals. The acoustic frequency device of this example can be incorporated into any of the appliances described in this disclosure.

### EXAMPLE 4

### Coated Elastomeric Hydraulic Dildo

The appliance in this example is similar to those in Examples 2 and 3, but is coated with a “skin-like” polyurethane. The coating is ideally applied to the fully inflated appliance so as not to be excessively taught during usage.

### EXAMPLE 5

### Variable Wall-Thickness Elastomeric Hydraulic Appliance

The appliance in this example is essentially like that of Example 2, but has a wall of varying thickness. When inflated the thinner parts of the wall protrude during the high-pressure phase of the hydraulic pressure cycle. If thin walled areas are radially asymmetrical, the appliance will bend and wiggle during the hydraulic pressure cycle.

### EXAMPLE 6

### Radially Restricted Elastomeric Hydraulic Appliance

The appliance of this example has an elastomeric wall which contains radially oriented fibers and/or rigid rings. The fibers and/or rigid rings restrict radial and not axial expansion. Therefore, during the hydraulic cycle the appliance elongates, but does not enlarge radially to any significant extent.

### EXAMPLE 7

### Helically Restricted Elastomeric Hydraulic Appliance

The appliance of this example has an elastomeric wall containing helically oriented fibers. During the hydraulic cycle this appliance twists in a manner consistent with the uncoiling action of the fiber helix.

### EXAMPLE 8

### Two-Sided Dildo with Hydraulic Oscillation

The two-sided dildo of U.S. Pat. No. 5,853,362, which uses a hook-shaped flexible dildo as an anchor (the internal dildo), can be modified to represent an example of the present invention. It is possible to make the external dildo, the internal dildo, or both vibrate via the time varying hydraulic pressure of the present invention. To do so, one or both dildos must be elastomeric and hollow, and be filled with hydraulic fluid that is connected via a tube or hose to a hydraulic driver. It is possible through use of flow restricting valves to attenuate the hydraulic actuation of either the internal or external dildo, so that unequal degrees of vibratory stimulation can be supplied to the wearer and her partner.

### EXAMPLE 9

### Two-Sided Dildo Intended to be Anchored in Both the Vagina and Anus with Hydraulic Oscillation

The two-sided dildo of U.S. Pat. No. 5,853,362 and Example 7, can be further modified by an additional hooked dildo connection to the wearer's anus. It may be desirable in this case to introduce a bending motion into the internal dildo so that the anal portion and the vaginal portion of said internal dildo are squeezed together as hydraulic pressure increases.

### EXAMPLE 10

### Penis Ring Attached to Hydraulic Mechanism to Vibrate the Erect Penis

The novel method of deforming portions of a sexual stimulator can be applied to the combination anal dildo/erection ring of U.S. Pat. No. 5,387,179. The application of time varying hydraulic pressure within a anal dildo/erection ring makes the device also function to vibrate the penis. It is also simple to make such a device also stimulate the prostate gland, for example with a hooked dildo to stimulate the prostate from the inside, as described in U.S. Pat. No. 5,853,362, or by rhythmically-variable pressure applied from outside the anal canal (via an inflating/deflating flexible cavity or sac that is in contact with the perineum between the scrotum and the anus), or even by gripping the prostate gently between two inflatable tubes so designed that they close around the prostate, in such a way that the grip varies in a stimulating manner.

### EXAMPLE 11

### Penis Ring which is Attached to Vibrating Mechanism to Surround the Scrotum and Testicles

Many heterosexual men are very hesitant to have any device anchored into their anus, as in Example 9. For such men, the idea of deriving pleasure from prostate or anal stimulation is abhorrent or at least uncomfortable. For this market segment, a vibrating cock ring can be designed to go around the penis and scrotum, so as to vibrate the erect penis by pulling it cyclically towards the scrotum. This is especially pleasurable for some women in rear entry or “doggie-style” vaginal sex, because the testicles are pulled into the woman's clitoris at the same time that the penis is pulled towards the testicles by the hydraulic vibration.

1. A hydraulically driven massager and/or sexual stimulator, linked to an hydraulic actuator capable of moving hydraulic fluid into and out of said hydraulically-driven sexual stimulator.

2. A hydraulically driven sexual stimulator of claim 1 in which the stimulator includes one or more electric, voice coil, or hydraulic motor driven vibrators.

3. A hydraulically driven sexual stimulator of claim 1 or 2 in which the stimulator includes at least one deformable, viscoelastic sac or tube which changes shape and/or hardness due to the injection and removal of hydraulic fluid by a suitable hydraulic actuator.

4. A hydraulically driven sexual stimulator of claim 3 in which fluid enters some or all of the deformable viscoelastic sexual stimulator elements along one path, and leaves said element through an independent path; this allows the control of temperature within the sexual stimulator element by controlling the temperature of the fluid which moves through the sexual stimulator.

5. A hydraulically driven sexual stimulator of claim 2, 3 or 4 in which the vibratory force applied to the stimulator consists of some combination of the following waveforms:
Low frequency oscillations having a frequency from approximately 0.1 to 5 Hertz, and involving a volume oscillation within said sexual stimulator of at least 10 ml; as a result of this oscillation some portion of the sexual stimulator changes length and/or circumference visibly during each cycle;
Higher frequency oscillations with relatively high frequencies of 5 Hertz or greater, and involving a volume oscillation within said sexual stimulator of no more than 5 ml of fluid per oscillation.
Non-sinusoidal and/or non-repetitive waveforms including but not limited to triangle waves, step waves, ramp waves, and repetitive pulses

6. A hydraulically driven sexual stimulator of claim 3, 4 or 5 in which a rhythmic oscillation is imposed upon an erect penis by inflatable fluid-filled viscoelastic tubes or sacs that surround or neighbor said erect penis and which expand and contract rhythmically because of movement of a hydraulic fluid in and out of said viscoelastic tubes or sacs.

7. A hydraulically driven sexual stimulator of claim 5 or 6 in which the amplitude, shape or phase of the coupled waveform is controlled partially or completely by signals derived from a biofeedback device of the prior art.

8. A hydraulically actuated dildo of claim 3, 4, 5 or 7 in which the hardness and degree of erection is controllable by the user.

9. A hydraulically driven sexual stimulator of claims 2, 3, 4, 5, 7, or 8 whose hydraulic sacs are so constructed as to produce thrusting motions, possibly coincident with an orifice.

10. A hydraulically driven sexual stimulator of claims 2, 3, 4, 5, 7, 8 or 9 in which successive hydraulic rings are connected by pressure controlled valves which are so engineered as to produce rhythmic waves when driven by a series of one or more hydraulic pressure pulses.

11. A hydraulically driven dildo of claim 10 in which the output of the last of a series of pressure driven valves is temporally coincident with the controlled release of pressurized fluid from the tip of the device.
